(19) United States
(12) Patent Application Publication
(10) Pub. No.: US 2021/0220269 A1
(43) Pub. Date: Jul. 22, 2021

(54) PHARMACEUTICAL COMPOSITION IN THE FORM OF A WATER-IN-OIL EMULSION (W/O) AND ITS USES

(71) Applicant: GALDERMA RESEARCH & DEVELOPMENT, Biot (FR)

(72) Inventors: Claire MALLARD, Mougins (FR); Gareth WINCKLE, Juan les Pins (FR); Emmanuelle GUTIERREZ, Saint-Laurent-du-Var (FR); Carole DUBAYLE, Mouans Sartoux (FR)

(21) Appl. No.: 17/214,389

(22) Filed: Mar. 26, 2021

Related U.S. Application Data
(63) Continuation of application No. PCT/EP2019/075876, filed on Sep. 25, 2019.

(30) Foreign Application Priority Data
Sep. 28, 2018 (FR) 18/01015
Sep. 28, 2018 (FR) 18/01017
Sep. 28, 2018 (FR) 18/01018

(51) Int. Cl. A61K 9/107; A61K 9/00; A61K 47/26; A61K 47/34; A61K 45/06

(52) U.S. Cl. CPC A61K 9/107; A61K 9/0014; A61K 45/06; A61K 47/34; A61K 47/26

(57) ABSTRACT

The invention relates to a composition in the form of a water-in-oil (W/O) emulsion suitable for topical administration. The invention is characterised by the fact that the composition is a composition comprises an aqueous phase representing 60% to 98% by weight of the composition and a fat phase comprising one or a plurality of oils and an emulsifying system

### CROSS-REFERENCE TO RELATED APPLICATION(S)

The present application is a continuation of and claims the benefit of priority to International Application No. PCT/EP2019/075876 filed Sep. 25, 2019, which claims the benefit of priority to French Patent Application No. 1801015 filed Sep. 28, 2018, to French Patent Application No. 1801017 filed Sep. 28, 2018, and to French Patent Application No. 1801018 filed Sep. 28, 2018, the entire contents of each of which are incorporated herein by reference.

### TECHNICAL FIELD

The present invention relates to the field of emulsions in the form of a water-in-oil emulsion and more preferably compositions, possibly pharmaceutical, suitable for topical or oral administration. It relates in particular to a composition in the form of an emulsion, as well as its cosmetic and food uses or a composition for its use as a medicinal product, more particularly in the prevention and/or treatment of dermatological diseases, in particular human skin diseases.

### PRIOR ART

Emulsions are used to carry both water-soluble and fat-soluble substances and are therefore particularly suitable for use in the food, cosmetics, pharmaceutical and veterinary sectors, and in the detergents sector.

In the field of topically applied pharmaceutical compositions, cosmetic or food compositions, there is a requirement on by the user of products in the form of emulsions to have emulsions that exhibit appropriate sensory characteristics. Emulsions that provide a feeling of freshness and that are felt especially when applied to the skin as not “sticky” are particularly sought after.

Emulsions are classified according to the nature of the continuous phase (also referred to as the “external phase”), in which droplets of the other phase (referred to as the “internal phase”) are dispersed.

In the case where the oil droplets are dispersed in a continuous aqueous phase, the system is called an “oil-in-water” (O/W) emulsion. 1

In the case where the water droplets are dispersed in a continuous oily phase, the emulsion is of the “water-in-oil” (W/O) type.

In general, among these two types of emulsions, it is easier to manufacture oil-in-water (O/W) type emulsions because W/O type emulsions are inherently thermodynamically unstable. Indeed, if equal quantities of water and oil are mixed, the formation of an aqueous continuous phase emulsion is always observed, because the cohesive forces between water molecules are stronger than those between oil molecules.

When these compositions are used for food purposes, W/O emulsions are advantageously used as spreads such as butter or margarine. Margarine is a W/O emulsion containing droplets of water or skimmed milk in a mixture of vegetable oils and fats.

Oily continuous phase (W/O) emulsions have many benefits:
the separation between water droplets reduces the possibility of the proliferation of microorganisms. The use of antiseptics, essential when the continuous phase is aqueous, can be avoided;
they can be stored well at low temperatures, being much less sensitive in this respect than H/W type emulsions;
the continuous oily phase covers the skin and protects it from dehydration and external substances.

In the development of an oily continuous phase emulsion system, two types of modifications were considered:
mechanical modifications concerning the combination of phases (order of addition of phases, flow control during phase combination, phase temperature, agitation speed, etc.);
chemical modifications, which consist of the addition of new ingredients or different contents resulting in the stabilisation of the emulsion.

With regard to mechanical modifications, the operating protocols used to prepare W/O emulsions generally require:
a significant energy supply in the form of thermal activation (aqueous and fat phases are typically heated to 80° C.), which must sometimes be followed by a well-controlled progressive cooling; and/or

the creation of turbulence in the two-phase emulsifying medium (high agitation speed (thousands of revolutions per minute) and high shear caused by specific agitator geometries).

With regard to chemical modifications, we can mention:
the use of microcrystalline waxes, such as ozokerite, which absorb the oil and prevent its exudation;
the use of liquid paraffins as a fat phase, as they are easier to emulsify;
the addition of mineral salts such as sodium chloride or magnesium chloride, in particular, to increase the cohesion of the interfacial film.

Document FR2852257 describes an emulsion consisting of an oily outer phase and a gelled aqueous phase, said aqueous phase representing 60 to 98% by weight, preferably 80 to 98% by weight, of the composition, as well as the process for manufacturing such an emulsion.

W/O emulsions with a very high proportion of aqueous phase (80 to 98%) have a sensory lightness and hydration associated with a cushion effect.

Some W/O emulsions marketed in particular by the company SEPPIC™ include:
an aqueous phase dispersed in very high concentration and requiring the presence of an electrolyte polymer; and
a continuous fat phase containing an emulsifying system such as those marketed under the name EASYNOV™ or FLUIDANOV™ and not necessarily requiring heating.

Unlike so-called conventional W/O emulsions, these compositions have the advantage of being prepared at room temperature depending on the melting points of the ingredients used. Indeed, emulsions are commonly prepared hot and their implementation generally requires complex preparation processes, a significant heating before emulsification as well as a precise control of the cooling process. In addition to being economical and simpler since it can be carried out at room temperature, the preparation process associated with these W/O emulsions requires only low shear, whereas high shear rates must generally be applied throughout the emulsification process.

In addition, this technology can be free from the use of water-soluble salts typically used in the aqueous phase for emulsion stabilisation.

In addition, it is not necessarily useful to add lipophilic thickeners (waxes) typically used to thicken the external fat phase to ensure good physical stability.

For this purpose, the emulsifying systems EASYNOV™ or FLUIDANOV™ necessarily include octyldodecyl xyloside, octyldodecanol and possibly PEG-30 DPHS (PEG-30 Macrogol 30 dipolyhydroxystearate or dipolyhydroxystearate), and the aqueous phase necessarily includes a polyelectrolyte type polymer.

Thus, W/O emulsions containing these emulsifying systems are necessarily intended for cosmetic application insofar as octyldodecyl xyloside does not belong to any pharmacopoeia.

The GELTRAP™ technology, for example, described by the company SEPPIC™, cannot be used for pharmaceutical application because the ready-to-use emulsifying mixture EASYNOV™ or FLUIDANOV™ are not registered in a monograph and do not have a pharmaceutical certification status.

The person skilled in the art seeking to develop new pharmaceutical compositions is therefore discouraged from using such emulsifying systems and cannot transpose the technical education associated with cosmetic compositions, in particular cosmetic W/O emulsions currently marketed, to the development of pharmaceutical compositions.

The use of W/O emulsions is a category of food products under development. Indeed, today's society is particularly sensitive to health problems conditioned by lifestyle, and in particular the dietary behaviour of individuals. Consumers seem to want to change their eating habits and lifestyle in order to preserve their health as much as possible. Faced with this change in consumer behaviour, the agri-food industry is seeking to develop new innovative products that promote people's health. Thus, newly marketed products often have their fat or fatty content reduced (more specifically in saturated fatty acids), in order to limit the risks of obesity, cardiovascular disease or diabetes, for example.

W/O emulsions, mainly represented by spreads such as butter and margarine, are major components of breakfast, and of food in general. Their sensory characteristics (melting, aroma, spreadability) are due to the physico-chemical properties of the lipids they contain and are therefore strongly linked to their fat content. The decrease in fat content in these foods results in the loss of the sensory qualities of these products, more specifically in the texture and firmness of the margarine.

This raises the problem of developing new spreads such as low-fat margarines, while maintaining their organoleptic properties, such as texture, firmness, melting profile, spreadability, mouth texture and taste, which ensure that the product is preserved under the recommended storage conditions.

For cosmetic use, the compositions of this invention are advantageously presented in the form of W/O emulsions with a very high proportion of aqueous phase (80 to 98%). They present a sensory effect of lightness and hydration combined with a cushion effect. And unlike the invention described in document FR2852257, the invention is preferably made with polymers on a natural basis.

### SUMMARY OF THE INVENTION

Thus, the present invention relates to the field of pharmaceutical, cosmetic or food compositions adapted for topical or oral administration, as well as their cosmetic and food uses. It also concerns a composition for its use as a medicinal product and more particularly for its use in the prevention and/or treatment of dermatological diseases, in particular human skin diseases.

### Technical Problem

Considering the above, a problem that the present invention proposes to solve is to develop:
a pharmaceutical composition comprising at least one component having a pharmaceutical certification status, said composition being in the W/O form highly concentrated in the aqueous phase and having in particular a sensory lightness and hydration associated with a cushion effect; but also
compositions improved to be in line with cosmetic trends based on the use of products on a natural basis and on dietary trends based on nutritional benefits, in the W/O form highly concentrated in the aqueous phase and presenting in particular a sensory effect of lightness and hydration combined with a cushion effect for cosmetic application, and in particular presenting an innovative texture combined with properties of use for food applications.

An additional problem that the invention proposes to solve is to develop compositions that are stable, economical, easy and quick to prepare.

### Technical Solution

The solution to this problem is first and foremost a composition in the form of a water-in-oil (W/O) emulsion comprising:
an aqueous phase representing 60% to 98% by weight of the composition, and
a fat phase comprising one or a plurality of oils and an emulsifying system.

Its second purpose is a pharmaceutical composition according to the invention, for its use in the prevention and/or treatment of dermatological diseases, in particular human skin diseases.

Its third purpose is the food use of a composition according to the invention.

Its fourth purpose is a process for preparing a pharmaceutical composition according to the invention, comprising the following steps of:
preparation of a fat phase comprising one or a plurality of oils, an emulsifying system, said system comprising at least one sorbitan ester and optionally a pharmaceutically active ingredient;
preparation, independently of the fat phase, of an aqueous phase comprising a polyelectrolyte polymeric rheology modifier and optionally a pharmaceutically active ingredient; at least one pharmaceutically active ingredient being present in the fat phase and/or the aqueous phase;
addition of the fat phase to the aqueous phase or vice versa; and
recovery of the pharmaceutical composition thus obtained.

Its fifth purpose is a process for the preparation of a cosmetic or food composition according to the invention, comprising the following steps:
preparation of a fat phase comprising one or a plurality of oils, a lipophilic emulsifying system,
preparation, independently of the fat phase, of an aqueous phase comprising a non-polyelectrolyte rheology modifier and/or a polyelectrolyte rheology modifier of natural origin;
addition of the fat phase to the aqueous phase or vice versa; and
recovery of the composition thus obtained.

Its last purpose is a process for preparing a pharmaceutical composition according to the invention, comprising the following steps of:
preparation of a fat phase comprising one or a plurality of oils, an emulsifying system, said system comprising at least one sorbitan ester and optionally a pharmaceutically active ingredient;
preparation, independently of the fat phase, of an aqueous phase comprising a non-polyelectrolyte rheology modifier, and optionally a pharmaceutically active ingredient; at least one pharmaceutically active ingredient being present in the fat phase and/or the aqueous phase;
addition of the fat phase to the aqueous phase or vice versa; and recovery of the pharmaceutical composition thus obtained.

### Advantages

In particular, the Applicant was able to develop pharmaceutical compositions that are highly concentrated water-in-oil (W/O) emulsions in aqueous phase (60% to 98% by weight of the total weight of the composition), which can be prepared cold and with low shear, and can incorporate pharmaceutical active ingredients of different chemical natures.

In addition, the compositions can be prepared cold and with low shear and can be advantageously prepared:
contain a relatively low fat content for food application unlike conventional W/O emulsions,
be made with polymers on a natural basis, and
be composed to support highly concentrated additives of interest for cosmetic application.

Unlike the so-called conventional W/O emulsions, the compositions associated with the present invention have the advantage of being able to reduce their percentage of fat without impacting the organoleptic properties.

In addition, when these compositions are used for cosmetic or pharmaceutical purposes, they have a high residual power after application to the skin, particularly thanks to their oily/fat external phase; they thus provide excellent protection against water loss and an emollient effect.

With a customisable texture, from liquid to more compact shapes, they are also adapted to different skin pathologies and different application sites such as the body, face, hands and feet.

These compositions according to the invention also have a soft and silky touch.

In addition, the compositions according to the invention are advantageously free of octyldodecanol and/or octyldodecylxyloside. The components used are pharmaceutical grade and allow the development of therapeutic compositions.

The Applicant proposes for this concept of composition formulation according to the invention, to use an aqueous phase rheology modifier which is a polyelectrolyte or non-polyelectrolyte polymer, a polyelectrolyte polymer of natural origin and/or a non-polyelectrolyte rheology modifier.

Consequently, the compositions invented make it possible to propose natural concepts and in particular to convey salified additives in high concentrations, which are frequent forms of water-soluble additives used in products such as cosmetics and food.

The Applicant was also able to demonstrate that the mode of action of non-polyelectrolyte polymers is not affected in the presence of ionic charges. The thickening of the aqueous phase always takes place.

In this description, unless otherwise specified, it is understood that, when an interval is given, it includes the upper and lower limits of that interval.

### DESCRIPTION

The invention concerns in particular a pharmaceutical composition which is a water-in-oil (W/O) emulsion suitable for topical administration. Said pharmaceutical composition therefore necessarily includes at least one pharmaceutically active ingredient or principle.

The active ingredient is preferably chosen from antibiotics, antibacterial agents, antivirals, antiparasitic agents, antifungals, anaesthetics, analgesics, painkillers, antiallergic agents, acneics, antimitotics, antipruritic drugs, antihistamines, immunosuppressants, corticosteroids, keratolytics, anti-angiogenes, anti-inflammatory agents including phosphodiesterase 4 inhibitors, anti-cancer drugs, anti-neoplastic drugs, natural extracts, anthracene derivatives, psoralens, anti-proliferative drugs (vitamin D analogues, etc.), anti-alopecics (prostaglandin analogues), anti-herpetics, photosensitisers, depigmentants, hormones, retinoids, vasoconstrictors and/or a mixture thereof.

As an example of a pharmaceutically active ingredient that can be used according to the invention, one can mention acetaminophen, acetylsalicylic acid, acitretin, azelaic acid, acyclovir, adapalene, alclometasone, alpha-tocopherol, amcinonide, amorolfine, amphotericin B, aprémilast, tetracycline, benzoyl peroxide, betamethasone, brimonidine, calcipotriol, calcitriol, ciclopirox, clindamycin, crisaborole, clobetasol, crotamiton, cyproheptadine, dapsone, desonide, diclofenac, diflucortolone, difluprednate, dioxyanthranol, econazole, efinaconazole, erythromycin, estradiol, etherinate, fluocinolone acetonide, fluticasone, fusidic acid, mometasone, glycolic acid, glycyrrhetinic acid, halobetasol, hydrocortisone, hydroquinone, ibuprofen, imiquimod, phosphodiesterase 4 (PDE4) inhibitors, mammalian target of rapamycin (mTOR) inhibitors, Janus kinase (JAK) inhibitors, isotretinoin, ivermectin, ketoconazole, kojic acid, lactic acid, lidocaine, malic acid, mequinol, methoxsalene, metronidazole, miconazole, minoxidil, octopirox, oxymetazoline, pilocaine, pyridoxine, progesterone, retinol, pimecrolimus, rapamycin, resiquimod, rucinol, tacrolimus, tazarotene, terbinafine, tetracaine, thenaldine, travopost, tretinoin, trimeprazine, trifarotene, zinc pyrithione, and salts or derivatives of these pharmaceutically active ingredients, taken alone or in a mixture.

Preferably, the pharmaceutically active ingredient is selected from azelaic acid, adapalene, amorolfine, benzoyl peroxide, brimonidine, calcipotriol, calcitriol, clindamycin, clobetasol, ivermectin, resiquimod, and salts or derivatives of these pharmaceutically active ingredients, taken alone or in a mixture.

More preferably still, the pharmaceutically active ingredient is chosen from adapalene, benzoyl peroxide, brimonidine, ivermectin, resiquimod, as well as their salts or derivatives, taken alone or in a mixture.

According to a particular embodiment of the invention, the composition includes a combination of adapalene and benzoyl peroxide. The Applicant was able to demonstrate that these two active ingredients, which are difficult to mix, were easily integrated into the compositions according to the invention.

According to a second specific embodiment of the invention, the composition comprises a pharmaceutically active ingredient chosen from a phosphodiesterase 4 inhibitor (PDE4), a mammalian target of rapamycin (mTOR) inhibitor, a Janus kinase (JAK) inhibitor, as well as salts or derivatives of these pharmaceutically active ingredients, taken alone or in a mixture.

The invention relates to a composition that is a water-in-oil (W/O) emulsion that includes a gelled aqueous phase and a fat phase.

The aqueous phase represents more than 50% by weight of the total weight of the composition and includes a polymeric rheology modifier of polyelectrolyte or non-polyelectrolyte type, a polyelectrolyte polymer of natural origin and/or a non-polyelectrolyte rheology modifier.

Preferably, the aqueous phase represents 60% to 98% by weight of the total weight of the composition.

More preferably still, the aqueous phase represents 80 to 95%, more preferably 75% to 90% by weight of the total weight of the composition.

The aqueous phase includes a rheology modifier.

An aqueous phase polymeric rheology modifier is any polymer that produces a modification of the aqueous phase in terms of rheology, in particular on flow profiles and viscosity of the aqueous phase. Thus, among others, gelling agents, viscosating agents, thickening agents, stabilising agents, suspending agents, texturising agents and film formers fall within this definition.

The rheology modifier of the composition is a polyelectrolyte polymer.

As an open-ended example of a rheology modifier that can be used in the composition according to the invention, one can mention synthetic polymers such as derivatives of acrylamide, acrylic acid and vinylpyrrolidone such as copolymers of acrylic acid and 2-acid-2-methyl-[(1-oxo-2-propenyl)amino] 1-propane sulfonic acid (AMPS), copolymers of acrylamide and 2-methyl-[(1-oxo-2-propenyl)amino]1-propane sulfonic acid, copolymers of 2-methyl-[(1-oxo-2-propenyl)amino]1-propane sulfonic acid and (2-hydroxyethyl)acrylate, the homopolymer of 2-methyl-[(1-oxo-2-propenyl)amino]1-propane sulfonic acid, the homopolymer of acrylic acid, the copolymers of acryloyl ethyl trimethyl ammonium chloride and acrylamide, the copolymers of AMPS and vinylpyrrolidone, copolymers of acrylic acid and alkyl acrylates whose carbon chain contains between ten and thirty carbon atoms, copolymers of AMPS and alkylacrylates whose carbon chain contains between ten and thirty carbon atoms. Polyelectrolyte polymers also include polymers of natural origin.

Examples include, but are not limited to, gelatin, carrageenans marketed under the name Viscarin™ GP by Danisco™, pectins marketed by Cargill™, alginates marketed by Danisco™, agarose, agar-agar, chitosan and xanthan gum known for example as Xanthan Gum™ FF sold by the company Jungbunzlauer™.

As an example of a polymeric rheology modifier that can be used preferentially in the composition according to the invention, one can mention in particular the products SEPINEO™ P600 (Acrylamide/Sodium Acryloyldimethyl Taurate Copolymer/Isohexadecane & Polysorbate 80), Carbopol ETD2020®, Pemulen TR1® and Pemulen TR2® Acrylates/C10-30 Alkyl Acrylate Crosspolymer, marketed by the company SEPPIC™.

More preferably, the polymeric rheology modifier of the composition according to the invention includes the polymeric rheology modifier marketed under the name SEPINEO™ P600 (Acrylamide/Sodium Acryloyldimethyl Taurate Copolymer/Isohexadecane & Polysorbate 80) by the company SEPPIC™, at contents up to 4% by weight of the total weight of the composition.

Non-polyelectrolyte rheology modifier refers to polymers that, when dissolved in a polar solvent such as water, dissociate in water, without causing charges to appear on their skeleton and counterions in solution, regardless of the pH evolution of the solution.

Thus, their behaviour is contrary to the behaviour of polyelectrolyte polymers which contain fillers on their skeleton either by simple dissolution (ionic polymers in aqueous solution) or by pH change.

The non-polyelectrolyte rheological modifier for pharmaceutical use is preferably chosen from:
non-polyelectrolyte polymers of natural origin such as guars, mannans, galactomanns, pullulan, dextran or inulin, starch and its derivatives such as starch acetate marketed under the name Beauty™ ST30 by the company Roquette™;
non-polyelectrolyte polymers of semi-synthetic origin such as cellulosic derivatives and in particular Natrosol™ 250 HHX marketed by the company Ashland™ and hydroxypropylcellulose such as Klucel™ MF marketed by Ashland™;
synthetic non-electrolyte polymers based on vinyl such as polyvinyl alcohol polymers and copolymers, polyvinyl pyrrolidone polymers and copolymers resold by Ashland™ under the Flexithix™ range;
synthetic non-polyelectrolyte polymers based on on acrylics, such as Acrylates/Behenet-25 Methacrylate/HEMA Crosspolymer-2 copolymers and in particular the product Carbopol SMART 3000™ offered by the company Lubrizol™.

According to a first advantageous embodiment of the invention, the composition includes a non-polyelectrolyte rheology modifier which is a polymer of semi-synthetic origin.

According to a second advantageous embodiment of the invention, the composition includes a non-polyelectrolyte rheology modifier which is a synthetic polymer based on vinyl.

According to a third advantageous embodiment of the invention, the composition includes a non-polyelectrolyte rheology modifier which is a synthetic polymer based on acrylics.

The rheology modifier of the cosmetic or food composition can be chosen from polymers of natural, animal or vegetable origin, such as:
gelatine,
casein,
albumin,
chitosan,
alginates,
agarose,
pectin,
agar-agar,
xylane,
scleroglucan,
amylose,
amylopectin,
starch and its derivatives,
starches modified with dextrins,
clays such as hectorites, bentonites, magnesium aluminium silicate, montmorillonite, and
mannans,
galactans,
pullulan,
dextran,
inulin,
natural gums of the galactomann family and others, such as guar gum, tara gum, curl gum, carrageenan gum, gellan gum, tragacanth gum, arabic gum or xanthan gum.

The rheological modifier is preferably chosen from:
non-polyelectrolyte polymers of natural origin such as guars, mannans, galactomanns, pullulan, dextran or inulin, starches and their derivatives such as starch acetate marketed under the name Beauty™ ST30 by the company Roquette™;
non-polyelectrolyte polymers of semi-synthetic origin such as cellulosic derivatives and in particular Natrosol™ 250 HHX marketed by the company Ashland™ and hydroxypropylcellulose such as Klucel™ MF marketed by Ashland™;
synthetic non-electrolyte polymers on a vinyl basis such as polyvinyl alcohol polymers and copolymers, polyvinyl pyrrolidone polymers and copolymers resold by Ashland™ under the Flexithix™ range;
synthetic non-polyelectrolyte polymers based on on acrylics, such as Acrylates/Behenet-25 Methacrylate/HEMA Crosspolymer-2 copolymers and in particular the product Carbopol SMART 3000™ offered by the company Lubrizol™.

According to a first advantageous embodiment of the invention, the composition includes a rheology modifier which is a non-polyelectrolyte polymer.

According to a second advantageous embodiment of the invention, the composition includes a rheology modifier which is a non-polyelectrolyte polymer of natural origin.

According to a third advantageous embodiment of the invention, the composition includes a non-polyelectrolyte rheology modifier which is a polymer of semi-synthetic origin.

According to a fourth advantageous embodiment of the invention, the composition includes a non-polyelectrolyte rheology modifier which is a synthetic polymer based on vinyl.

According to a fifth advantageous embodiment of the invention, the composition includes a non-polyelectrolyte rheology modifier which is a synthetic polymer based on acrylics.

In addition, when these compositions are used for food, the aqueous phase may also include a variety of water-soluble ingredients, which include sugars, acids, bases, proteins, carbohydrates, surfactants.

In addition, when these compositions are used for cosmetic purposes, the aqueous phase may also include additives such as humectants, preservatives, dyes, perfumes, mineral fillers, synthetic fillers, surfactants and any other cosmetic additive added, alone or in a mixture, to affect the protection, appearance, balance and regeneration of the skin. These include sunscreens, mineral salts, trace elements, fruit acids, plant extracts and antioxidants.

The Applicant was also able to demonstrate that glycerol, integrated into the initial formula, could be substituted by propylene glycol, which could possibly be a better solvent for the pharmaceutical active ingredient, or by any other glycol that has an anti-freeze role. The Applicant has thus developed a composition comprising brimonidine and 5% propylene glycol by weight of the total weight of the composition.

As an example, glycols (glycerin, propylene glycol, PEG 400, sorbitol, isosorbide) could be tested by the Applicant at levels of 4 to 34% by weight of the total weight of the composition to determine the limits of the prepared compositions.

The aqueous phase may also include hydrophilic solvents, which, among other things, act as solubilisers of pharmaceutical active ingredients or as propensants. One example is dimethyl sulfoxide (Procipient DMSO™ marketed by Gaylord Chemical™), for example at a concentration of 30% by weight of the total weight of the composition.

Diethylene glycol monoethyl ether (Transcutol HP™ sold by the company Gattefossé™) can also be used up to 25%, for example.

In addition, ethanol may also be present as a solvent, up to about 30% by weight of the total weight of the composition, in order to help, for example, preserve the composition according to the invention in the presence of preservatives. For the same purpose, hexylene glycol can also be used, for example at a concentration of 10% by weight of the total weight of the composition.

The fat phase of the composition according to the invention includes one or a plurality of oils, as well as an emulsifying system.

The oil or oils usable in the composition according to the invention may be present up to 30% by weight of the total weight of the composition.

When pharmaceutical use is made of the compositions, usable oils include mineral, vegetable or animal oils. Examples of vegetable oils include sweet almond oil, avocado oil, castor oil, olive oil, jojoba oil, sunflower oil, wheat germ oil, sesame oil, groundnut oil, grape seed oil, soybean oil, rapeseed oil, safflower oil, copra oil, corn oil, hazelnut oil, shea butter, palm oil, apricot kernel oil, calophyllum oil; as animal oil, perhydrosqualene; as mineral oils, paraffin oil and vaseline oil; and mixtures thereof.

By way of example, the following products can be mentioned as examples of oil, i.e. emollient that can be used preferentially:
PRISORINE™ 3505 LQ (isostearic acid);
PRISORINE™ 3515 LQ (isostearyl alcohol);
KOLLICREAM™ OD (octyldodecanol);
KOLLICREAM™ OA (Oleyl alcohol);
SCHERCEMOL™ DIA ESTER (Diisopropyl adipate);
MIGLYOL™ 812N (Caprylic/capric triglycerides);
CRODAMOL™ IPIS (Isopropyl isostearate);
CRODAMOL™ AB (C12-15 Alkyl Benzoate);
CRODAMOL™ IPP (Isopropyl palmitate);
CRODAMOL™ IPM (Isopropyl myristate);
SCHERCEMOL™ 1688 (Cetearyl ethyl hexanoate);
CERAPHYL™ 41 (C12-15 Alkyl lactacte);
CERAPHYL™ 31 (Lauryl lactate);
CRODAMOL™ ML (Myristyl lactate);
CRODAMOL™ EO (Ethyl oleate);
KOLLICREAM™ DO (Decyl oleate);
CRODAMOL™ OO (oleyl oleate);
LIPO™ PGO-3 (polyglyceryl-3 oleate);
ARLAMOL™ PS11E-LQ (PPG-11 Stearyl ether) ;
SILKLFLO™ 366 (hydrogenated polyisodecene);
MARCOL™ 52 (mineral oil);
MARCOL™ 82 (mineral oil);
PRIMOL™ 352 (mineral oil);
PARLEAM™ (hydrogenated polyisobutene);
Sweet almond oil;
Olive oil
SQUALANE™ PE (Squalane); taken alone or in a mixture.

In addition, when these compositions are used for food, the oil phase may also include triglycerides, diglycerides, monoglycerides, free fatty acids, sterols and vitamins.

When a cosmetic use is made of these compositions the oily phase may also include one or more oils chosen from among:
vegetable oils, such as sweet almond oil, copra oil, monoi oil, castor oil, jojoba oil, olive oil, rapeseed oil, groundnut oil, sunflower oil, wheat germ oil, corn germ oil, soybean oil, cottonseed oil, lucerne oil, poppy seed oil, pumpkin oil, evening primrose oil, millet oil, barley oil, rye oil, safflower oil, bankoulier oil, passionflower oil, hazelnut oil, palm oil, shea butter, apricot kernel oil, calophyllum oil, sysymbrium oil, avocado oil, calendula oil;
vegetable oils and their ethoxylated methyl esters;
oils of animal origin such as squalene, squalane;
mineral oils such as paraffin oil, vaseline oil and isoparaffins;
synthetic oils, including fatty acid esters such as butyl myristate, propyl myristate, cetyl myristate, isopropyl palmitate, butyl stearate, hexadecyl stearate, isopropyl stearate, octyl stearate, isoketyl stearate, dodecyl oleate, hexyl laurate, propylene glycol dicaprylate, lanolic acid esters, such as isopropyl lanolate, isoketyl lanolate, monoglycerides, diglycerides and triglycerides of fatty acids such as glycerol triheptanoate, alkylbenzoates, polyalphaolefins, polyolefins such as polyisobutene, synthetic isoalkanes such as isohexadecane, isodecane, perfluorinated oils and silicone oils. These include in particular dimethylpolysiloxanes, methylphenylpolysiloxanes, amine-modified silicones, fatty acid-modified silicones, alcohol-modified silicones, alcohol- and fatty acid-modified silicones, polyether-modified silicones, epoxy-modified silicones, fluorinated group-modified silicones, cyclic silicones and alkyl group-modified silicones.

Preferably, one can mention in particular:
as vegetable oils, sweet almond oil, avocado oil, castor oil, olive oil, jojoba oil, sunflower oil, wheat germ oil, sesame oil, peanut oil, grape seed oil, soybean oil, rapeseed oil, safflower oil, copra oil, corn oil, hazelnut oil, shea butter, palm oil, apricot kernel oil, calophyllum oil;
as an oil of animal origin, perhydrosqualene;
as mineral oils, paraffin oil and vaseline oil.

The oils that can be used in cosmetic compositions according to the invention are preferably present in a concentration of between 2 and 40%, preferably between 2 and 20% by weight of the total weight of the composition.

In addition to emollients, the fat phase may also contain waxes, including beeswax called Cerabeil bleached Dab™ marketed by Baerlocher™. These waxes at contents of 0.5% by weight of the total weight of the composition as an example are added as a consistency factor and therefore make it possible to ensure better stability of the compositions in the case of the use of emollients known to be difficult to formulate in this technology or in the presence of pharmaceutically active ingredients if they are found to destabilise the composition.

Other consistency factors than beeswax could be used in the fat phase, in particular modified waxes such as Stearoxy Trimethylsilane (and) stearyl alcohol, referred to as Silky wax ¹⁰™ by Dow Corning™, for example at a concentration of 1% by weight of the total weight of the composition.

The raw material ST-Elastomer 10™ from Dow Corning™, named INCI Cyclopentasiloxane (and) Dimethicone Crosspolymer, was also used at 1% by the Applicant in an example of composition according to the invention. One can also mention silicas, conventional colloidal silicon dioxide with with silica dimethyl silylate referenced under the trade name Aerosil R972™from Evonik™, used at 0.5% by weight of the total weight of the composition in an example of composition according to the invention.

Polyolprepolymer-2 (PPG-12/SMDI Copolymer) from Mylan Bertek Pharmaceuticals™ can also be used, for example at a concentration of 1% by weight of the total weight of the composition.

Glyceryl dibehenate is a thickener known for its fat phase and can also be used. In particular, the Applicant integrated the glyceryl behenate marketed under the name Compritol™ 888 by Gattefossé™ into a concentration of 1% by weight of the total weight of the composition.

Tribehine and its derivatives including glyceryl dibehenate which is a thickener known for its fat phase and can also be used. In particular, the Applicant integrated the glyceryl behenate marketed under the name Compritol™ 888 by Gattefossé™ into a concentration of 1% by weight of the total weight of the composition.

Synthetic copolymer-based gelling agents such as TRANSGEL™ from AIGLON™, VERSAGEL™ from CALUMET™, polyam ides OLEOCRAFT™ from CRODA™, the NOMCORT™ range from NISSHIN OILLIO™.

Modified clays can also be used, such as TIXOGELS™ from BYK™, BENTONES™ from ELEMENTIS SPECIALITIS™.

The fat phase of pharmaceutical compositions also includes an emulsifying system that incorporates at least one sorbitan ester, which allows the emulsion to be formed quickly.

Sorbitan esters form a class of non-ionic surfactants derived from sorbitan by esterification of one or a plurality of its alcohol or phenol functions. Sorbitan esters are sometimes referred to as SPAN™.

As an example, without limitation, of sorbitan esters that can be used in the composition according to the invention, the following products may be mentioned:
sorbitan monostearate (SPAN™ 60);
sorbitan tristearate (SPAN™ 65);
sorbitan monolaurate (SPAN™ 20);
sorbitan monooleate (SPAN™ 80);
sorbitan monopalmitate (SPAN™ 40);
sorbitan trioleate (SPAN™ 85).

The sorbitan esters chosen allow the development of pharmaceutical compositions according to the invention.

Preferably, the sorbitan ester used is sorbitan monooleate (SPAN™ 80), present up to contents of 4% by weight of the total weight of the composition.

Advantageously, the emulsifying system of pharmaceutical compositions also includes a lipoamino acid or a salt thereof, a lipopeptide or a salt thereof, a polyglycerol ester or a glycerol stearate.

Preferably, the emulsifying system of pharmaceutical compositions includes a polyglycerol ester such as:
macrogol 30 dipolyhydroxystearate (PEG-30 dipolyhydroxystearate) marketed under the name CITHROL DPHS by the company CRODA™; at contents up to 1% by weight of the total weight of the composition;
polyglyceryl-4 isostearate marketed under the name ISOLAN™ GI 34 by Evonik Nutrition & Care GmbH™;
polyglyceryl-3 diisostearate marketed under the name Plurol™Diisostearic CG by the company Gattefossé™.

Particularly preferably, the emulsifying system of pharmaceutical compositions includes a polyglycerol ester which is macrogol dipolyhydroxystearate 30 which is available in a pharmaceutical grade. In particular, it allows the emulsion interface to be stabilised due to steric obstruction.

Where the pharmaceutical combination that is the object of the invention includes a combination of sorbitan ester and polyglycerol ester, the Applicant was able to demonstrate that a higher concentration of sorbitan ester is preferred. Thus, the ratio of sorbitan ester/polyglycerol ester is advantageously between 2:1 and 10:1, preferably between 3:1 and 5:1, preferably again this ratio is about 4:1.

According to a particularly preferred embodiment of the invention, the pharmaceutical composition comprises sorbitan oleate/PEG-30 dipolyhydroxystearate in a ratio of 4:1.

These two components of the emulsifying system according to the invention are preferred in particular insofar as sorbitan oleate has a USP and EP monograph and PEG-30 dipolyhydroxystearate has an EP monograph.

In addition, the pharmaceutical composition that is the object of the invention is advantageously free of octyldodecanol and/or octyldodecylxyloside.

The Applicant was able to evaluate the impact of octyldodecanol on the compositions that were the subject of the invention. It appears that the incorporation of octyldodecanol causes more destabilisation than stabilisation assistance.

In addition, the composition that is the object of the invention is advantageously free of octyldodecylxyloside which does not belong to any pharmacopoeia.

The fat phase of cosmetic or food compositions also includes a lipophilic emulsifying system comprising one or a plurality of emulsifying surfactants.

Emulsifying surfactants for cosmetic or food compositions that may be used in the context of this invention include lipoamino acids and their salts; lipopeptides and their salts; sorbitan esters such as the product marketed under the name MONTANE™ 80 by the company SEPPIC™; polyglycerol esters such as the products marketed under the name ISOLAN™ GI34 by BASF™ and PLUROL™ DIISOSTEARIQUE by GATTEFOSSE™; ethoxylated castor oil and ethoxylated hydrogenated castor oil, such as the product marketed under the name SIMULSOL™ 989 by the company SEPPIC™; glycerol stearate; polyglycol or polyglycerol polyhydroxystearates, such as the products HYPERMER™ B246, ARLACEL™ P135 marketed by the company UNIQEMA™, the product DEHYMULS™ PGPH marketed by the company COGNIS™, the product DECAGLYN™ SHS marketed by the company NIKKO™; polyethylene glycol-alkylglycol copolymers such as PEG-45 dodecylglycol copolymer such as the product marketed under the name ELFACOS™ ST 9 by the company AKZO™, ethoxylated sorbitan esters such as products marketed under the name MONTANOX™ by the company SEPPIC™; low-ethoxylated protein acylates (from 1 to 3 OE groups); ethoxylated beeswax such as the product named APIFIL™ marketed by the company GATTEFOSSE™; cationic emulsifiers such as aminoxides, quaternium 82 and surfactants described in patent application WO96/00719 and mainly those with at least 16 carbon atoms in the fatty chain; sucrose esters, methylglucoside esters, ethoxylated or not; ethoxylated fatty acids; ethoxylated fatty alcohols; anionic emulsifiers such as decylphosphate or cetarylsulphate; aluminium polyoxystearate, such as for example the product marketed under the name MANALOX™ marketed by the company RHODIA™; magnesium stearate; aluminium stearate.

Non-ionic and anionic silicone emulsifying surfactants are also likely to be used in this invention for cosmetic or food compositions.

It is also possible to use emulsifying surfactants of the alkyl polyglycoside type, for example those described in patent application FR-A-790977, in particular xylose derivatives for cosmetic or food compositions.

It is also possible to use for advantageously cosmetic or food compositions an emulsifier based on alkyl polyglycosides and fatty diols, including in particular:
5 to 95 parts by weight of an alkyl polyglycoside mixture consisting of the reaction products of a saccharide and a dimerdiol having 36 carbon atoms;
95 to 5 parts by weight of a dimerdiol having 36 carbon atoms. Preferred emulsifiers, as defined above, include:
5 to 60 parts by weight of the above-mentioned mixture of alkyl polyglycosides; and
95 to 40 parts by weight of dimerdiol having 36 carbon atoms.

The alkyl polyglycoside mixture consisting of the reaction products of a saccharide and a dimerdiol having 36 carbon atoms is actually a mixture in any proportion of hydroxyalkyl polyglycosides (products resulting from the acetalisation of one of the two hydroxyl groups of the dimerdiol) and polyglycosylalkylpolyglycosides (products resulting from the acetalisation of the two hydroxyl groups of the dimerdiol).

These alkyl polyglycosides can be represented by the following formulae I and II respectively:

HO—R—O-(G)ₙ (I)

(G)ₘ-OR—O-(G)ₚ (II)

where: G represents a saccharide residue; R represents a disubstituted group derived from dimer alcohol derived from the hydrogenation of dimer acid; n, m and p represent the average degree of polymerisation of each saccharide residue.

The product known as “dimeric acid” is a dibasic acid having 36 carbon atoms, the majority compound of which can be represented by the formula:

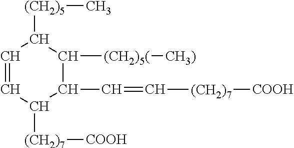

The above-mentioned alkyl polyglycosides may contain, as sucrose residue, a glucose or dextrose residue, fructose, galactose, mannose, ribose, xylose, preferably a glucose or xylose residue.

It should also be noted that each unit of the polyoside part of the above-mentioned alkyl polyglycosides may be in anomerical form α or β, and the rest of the saccharide can be furanoside or pyranoside.

The average degree of polymerisation of each saccharide residue is generally between 1.05 and 2.5, preferably between 1.1 and 2.

The term “alkylpolyglycoside” used in this application therefore refers to either alkylmonooside (degree of polymerisation equal to 1) or alkylpolyglycoside (degree of polymerisation greater than 1).

The dimerdiol used for the preparation of the emulsifying surfactant above is a diol derived from the hydrogenation of dimer acid.

It is marketed by the Company COGNIS™ under the name SPEZIOL™ C 36/2.

This compound, due to its origin, may contain minor proportions of impurities. Such impurities may be present in amounts up to 30% by weight of the total weight of diol.

Therefore, emulsifying surfactants based on alkyl polyglycosides and fatty diols may include, in corresponding minor proportions, such impurities, or the reaction products of such impurities with a saccharide. Emulsifying surfactants based on alkyl polyglycosides and fatty diols that can be used in this invention can be prepared by simply mixing their constituents in desired predetermined proportions.

On an industrial scale, they should preferably be prepared according to one of the two methods traditionally used for the synthesis of alkyl polyglycosides, and for example by reaction, in an acid medium, between dimerdiol and a saccharide having an anomeric OH, such as glucose or dextrose.

If necessary, this synthesis may be supplemented by operations of neutralisation, filtration, distillation or partial extraction of the excess fatty diol or discolouration.

It may also be particularly advantageous to use an alkylpolyxyloside emulsifying surfactant, as described in application EP-A-1142901, of formula:

R—O—(X)ₚ
where p represents a decimal number between 1 and 5, X represents the remainder of the xylose, and R represents a branched alkyl radical:

CH (CₙH₂ₙ₊₁)(CₘH₂ₘ₊₁)—CH₂
where m is an integer from 6 to 18, n is an integer from 4 to 18 and the sum of n+m is greater than or equal to 14;
or, in a particularly preferential embodiment, a composition consisting of a mixture of at least two compounds as defined above;
or a composition comprising more than 0% by weight and less than 100% by weight, preferably from 1% to 60% by weight, of a compound or mixture of compounds defined above and more than 0% by weight and less than 100% by weight, preferably from 40% to 99% by weight, of a compound or mixture of compounds of the formula ROH in which R has the meaning mentioned above.

Particularly advantageously, a mixture of alkylpolyxyloside R-0-(X)p and its corresponding alcohol ROH is used in the proportions indicated above.

For cosmetic or food compositions, an emulsifying system containing at least one emulsifying surfactant chosen from alkylpolyglycosides, alkylpolyglycoside and fatty alcohol compositions, polyglycerol or polyglycol esters or polyol esters such as polyglycol or polyglycerol polyhydroxystearates is used.

Even more advantageously, an emulsifying system containing a polyol polyhydroxystearate or a polyglycerol ester, in combination with an alkylpolyglycoside and fatty alcohol composition, is used for cosmetic or food compositions.

Emulsions of cosmetic or food compositions may preferably contain up to 10% by weight of a co-emulsifier.

Co-emulsifiers of cosmetic or food compositions that may be used in this invention include lipoamino acids and their salts, lipopeptides and their salts, sorbitan esters, polyglycerol esters, ethoxylated hydrogenated castor oil, glycerol stearate, cationic emulsifiers such as aminoxides, quaternium 82, sucrose esters, methylglucoside esters, ethoxylated or non-ethoxylated fatty acids, ethoxylated fatty alcohols, anionic emulsifiers such as decylphosphate or cetarylsulfate.

Non-ionic and anionic silicone emulsifying surfactants are also likely to be used as co-emulsifiers for cosmetic or food compositions according to the invention.

In addition, the fat phase may also include lipophilic solvents and any other lipophilic compounds such as preservatives or perfumes.

In addition, the interfacial zone between the aqueous and fat phases may contain a variety of non-restrictive compounds such as amphiphilic compounds such as proteins, phospholipids, surfactants, alcohols and compounds in the form of solid particles.

The ingredients of the aqueous phase, the fat phase, and the interfacial zone can create microstructures within these regions under the appearance of fat crystals, aggregates, air bubbles, liquid crystals and micelles, for example.

Phenoxyethanol is advantageously an integral part of the fat phase of pharmaceutical compositions. Sometimes, and this is the case in particular for the active ingredient Ivermectin, phenoxyethanol with a content of 1% by weight of the total weight of the composition plays a role as a solubiliser of the active ingredient while intervening in the preservation of the composition.

Arlasolve DMI (dimethyl isosorbide) can also be present in formulation as a solubiliser of active ingredients, for example at a content of 5% by weight of the total weight of the composition.

The pharmaceutical composition which is the object of the invention is particularly suitable for its use in the prevention and/or treatment of dermatological diseases, in particular human skin diseases.

More particularly, the pharmaceutical composition which is the object of the invention is suitable for use in the prevention and/or treatment of dermatological conditions, in particular human skin diseases defined below:
dermatological conditions linked to a keratinisation disorder relating to cell differentiation and proliferation, in particular to treat vulgar, comedonian, polymorphic, rosaceous, nodulocystic, conglobata, senile and secondary acne such as solar, medicinal or professional acne;
keratinisation disorders, including ichthyosis, ichthyosiform states, lamellar ichthyosis, Darrier's disease, palmoplantar keratodermies, leukoplakia, pityriasis rubra pilaris and leukoplasiform states, cutaneous or mucous lichen (buccal);
dermatological conditions with an inflammatory immuno-allergic component, with or without cell proliferation disorders, and in particular all forms of psoriasis, whether skin, mucous membrane or nail, and even psoriatic arthritis, or atopic dermatitis and the different forms of eczema;
skin disorders due to exposure to UV radiation as well as to repair or fight against skin ageing, whether photo-induced or chronological or to reduce pigmentations and actinic keratoses, or any pathologies associated with chronological or actinic ageing, such as xerosis, pigmentations and wrinkles;
any condition related to benign dermal or epidermal proliferation, whether or not of viral origin, such as vulgar warts, flat warts, molluscum contagiosum and epidermodysplasia warts, oral papillomatoses or florids;
dermatological disorders such as immune dermatoses such as lupus erythematosus, bullous immune diseases and collagen diseases such as scleroderma;
stigmas of epidermal and/or dermal atrophy induced by local or systemic corticosteroids, or any other form of skin atrophy,
healing disorders, or to prevent or repair stretch marks, or to promote healing,
in the treatment of any microbial, viral and fungal disease at the skin level such as tinea pedis and tinea versicolor,
pigmentation disorders, such as hyperpigmentation, melasma, hypopigmentation or vitiligo; and
cancerous or precancerous, cutaneous or mucosal conditions and their effects and side effects (e.g. oncodermatology) such as actinic keratoses, Bowen's disease, in-situ carcinomas, keratoacanthoma and cutaneous cancers such as basal cell carcinoma (BCC), squamous cell carcinoma (SCC) and tek skin lymphomas such as T lymphoma.

In other words, the invention also relates to a pharmaceutical composition should the invention be used as a medicinal product in the treatment of dermatological diseases, in particular human skin diseases, as previously defined.

The invention also covers the cosmetic use of the composition according to the invention. Preferably, the composition used is applied topically or orally, preferably topically.

The invention still has as its object the food use of the composition according to the invention. The composition used as a food composition is administered orally.

A food composition is a composition that can be used in the diet of a mammal. Advantageously, the food composition is in the form of liquid, semi-solid or solid. Advantageously, the food composition is in liquid form, such as a sauce or liquid butter; semi-solid or solid, such as butter or ice cream, a spread.

The composition also generally contains a number of additives such as carbohydrates, synthetic essential amino acids, minerals and vitamins. Suitable carbohydrates are starch, lactose, sucrose, fructose, dextrose or a mixture of these.

Advantageously, the dietary composition includes vitamins such as vitamin A, B1, B2, B2, B5, B6, B8, B9, B12, C, D, E, K, PP.

Typically, the food composition also includes trace elements and/or minerals, such as selenium, zinc or copper.

Advantageously, the food composition also includes one or more ingredients chosen from prebiotics, probiotics, co-enzyme Q10, antioxidants, texturising agents, colourants, thickeners, flavours, or a mixture thereof.

The invention relates to the use of the composition according to the invention as a food supplement or in nutritional programmes of renutrition, or nutritional supplementation, or to compensate for the deficiencies of adults, athletes, the elderly, or persons in need of improvement of their physical condition, such as sick, bedridden, weak, undernourished or sarcopenic persons.

Typically, according to the invention, maintaining or improving physical fitness includes improving muscle performance, maintaining muscle mass, improving muscle synthesis, improving physical performance and fatigue resistance, improving physical mobility, improving renutrition response and preserving bone density.

Another purpose of the invention is to create a method for preparing a pharmaceutical composition according to the invention comprising the following steps:
preparation of a fat phase as described above comprising one or a plurality of oils, an emulsifying system, said system comprising at least one sorbitan ester and optionally a pharmaceutically active ingredient;
preparation, independently of the fat phase, of an aqueous phase as described above comprising a polyelectrolyte polymeric rheology modifier and optionally a pharmaceutically active ingredient; at least one pharmaceutically active ingredient being present in the fat phase and/or the aqueous phase;
addition of the fat phase to the aqueous phase or vice versa; and
recovery of the pharmaceutical composition thus obtained.

Preferably, in the last step of the manufacturing process according to the invention, the fat phase is added to the aqueous phase.

Another purpose of the invention is to create a method for preparing a composition according to the invention comprising the following steps:
preparation of a fat phase comprising one or a plurality of oils, a lipophilic emulsifying system,
preparation, independently of the fat phase, of an aqueous phase comprising a non-polyelectrolyte rheology modifier and/or a polyelectrolyte rheology modifier of natural origin;
addition of the fat phase to the aqueous phase or vice versa; and
recovery of the composition thus obtained.

Preferably, in the last step of the manufacturing process according to the invention, the fat phase is added to the aqueous phase.

Finally, the last purpose of the invention is a method for preparing a pharmaceutical composition according to the invention, comprising the following steps of:
preparation of a fat phase comprising one or a plurality of oils and lipophilic emulsifying system and optionally a pharmaceutically active ingredient;
preparation, independently of the fat phase, of an aqueous phase comprising a non-polyelectrolyte rheology modifier, and optionally a pharmaceutically active ingredient; at least one pharmaceutically active ingredient being present in the fat phase and/or the aqueous phase;
addition of the fat phase to the aqueous phase or vice versa; and
recovery of the pharmaceutical composition thus obtained. Preferably, in the last step of the manufacturing process according to the invention, the fat phase is added to the aqueous phase.

### EXAMPLES

The present invention will now be illustrated by the following examples. For all examples, the asterisk “*” indicates that the percentages are given by weight of the total weight of the composition (w/w).

### Example 1

### Comparison of Water/Oil (W/O) Emulsions Obtained Using Different Emulsifiers

The detailed compositions of compositions A and B are described in Tables 1 and 2 respectively below.

TABLE 1
Formula A which is a cosmetic W/O composition
comprising Octyldodecylxyloside (positive control)

| INCI name | % w/w* |
|---|---|
| Octyldodecanol Octyldodecylxyloside | 3.0 |
| PEG-30 Dipolyhydroxystearate | |
| (EASYNOV ™) | |
| Caprylic capric triglycerides | 10.0 |
| Phenoxyethanol | 0.4 |
| Cyclomethicone | 5.0 |
| Glycerol | 6.0 |
| Methyl Parahydroxybenzoate | 0.1 |
| Acrylamide, AMPS dispersion | 4.0 |
| copolymer 40% isohexadecane | |
| Water | 71.5 |

*The above percentages are given by weight of the total weight of the composition (w/w)

TABLE 2
Formula B which is an W/O composition suitable
for pharmaceutical use according to the invention

| INCI name | % w/w* |
|---|---|
| Sorbitan Monooleate | 1.2 |
| Macrogol 30 Dipolyhydroxystearate (PEG-30 DPHS) | 0.3 |
| Caprylic capric triglycerides | 10.0 |
| Phenoxyethanol | 0.4 |
| Cyclomethicone | 5.0 |
| Glycerol | 6.0 |
| Methyl Parahydroxybenzoate | 0.1 |
| Acrylamide, AMPS dispersion copolymer 40% | 4.0 |
| isohexadecane | |
| Water | 73.0 |

Formulas A and B were compared. The results are presented in Table 3 below.

TABLE 3
Characterisation and stability results

| | Formula A | Formula B |
|---|---|---|
| Macroscopic appearance | White cream that flows | White cream that flows |
| Microscopic appearance | Fine Fraction globules | Fine Fraction globules |
| (Zeiss Axio Scope | <10 μm | <10 μm |
| A1 Microscope) | | |
| Viscosity | RV/S04/5 rpm-for 1 | RV/S04/5 rpm-for 1 |
| | min. | min. |
| Brookfield DVII + Pro | 22,440 cP (56%) | 25,760 cP (64%) |
| Stability (+40° C.) | >3 months | >3 months |

The above results show that the physical characteristics of the two formulations are similar. The combination of sorbitan oleate and macrogol 30 dipolyhydroxystearate (PEG-30 DPHS) as an emulsifier couple in formulation B results in an emulsion with particularly beneficial physico-chemical characteristics.

Formulations are stable for at least three months at +40° C., without modification of the physical components, i.e. macroscopic observation, microscopic observation and viscosity.

### Example 2

### Water/oil (W/O) Emulsion Compositions Suitable for Pharmaceutical Use According to the Invention Comprising Pharmaceutically Active Ingredients

The formulae C and D detailed below are obtained under low shear and in the presence of an emulsifying couple consisting of sorbitan monooleate and macrogol 30 dipolyhydroxystearate (PEG-30 DPHS).

TABLE 4
Formula C which is a W/O composition that contains an active
ingredient in the external fat phase: Solubilised Ivermectin

| INCI name | % w/w* |
|---|---|
| Sorbitan Monooleate | 4.0 |
| Macrogol 30 Dipolyhydroxystearate (PEG-30 DPHS) | 1.0 |
| beeswax | 0.5 |
| Caprylic capric triglycerides | 5.0 |
| Disopropy adipate | 5.0 |
| Phenoxyethanol | 1.0 |
| Propyl Parahydroxybenzoate | 0.1 |
| Active ingredient: Ivermectin | 1.0 |
| Glycerol | 4.0 |
| Methyl Parahydroxybenzoate | 0.2 |
| Acrylamide, AMPS dispersion copolymer 40% | 4.0 |
| isohexadecane | |
| Water | 74.2 |

The characterisation and stability results of formula C are shown in Table 5 below.

TABLE 5
Characterisation and stability results

| | Formula C |
|---|---|
| Macroscopic appearance | Semi-fluid white cream |
| Microscopic appearance | Fine fractionation, globules <2.5 μm |
| Zeiss Axio Scope Al | mainly and some rare globules <15 μm |
| Microscope | Slight refractions of beeswax |
| Viscosity Brookfield DVII + | RV/SSA/S27/6 rpm-at 1 min |
| Pro | 21,083 cP (51%) |
| Physical stability of the | 3 MONTHS |
| formulation (+40° C.) | |
| Chemical stability of the | >3 MONTHS |
| active ingredient in the | |
| formulation (+40° C.) | |

Formulation C is homogeneous and could be prepared.

TABLE 6
Formula D which is a W/O composition that
contains two active ingredients in the external fat
phase: Solubilised Ivermectin and dispersed Adapalene

| INCI name | % w/w* |
|---|---|
| Sorbitan Monooleate | 4.0 |
| Macrogol 30 Dipolyhydroxystearate (PEG-30 DPHS) | 1.0 |
| beeswax | 0.5 |
| Caprylic capric triglycerides | 5.0 |
| Disopropyl adipate | 5.0 |
| Phenoxyethanol | 1.0 |
| Propyl Parahydroxybenzoate | 0.1 |
| Active ingredient 1: Ivermectin | 1.0 |
| Active ingredient 2: Adapalene | 0.3 |
| Glycerol | 4.0 |
| Methyl Parahydroxybenzoate | 0.2 |
| Acrylamide, AMPS dispersion copolymer 40% | 4.0 |
| isohexadecane | |
| Water | 73.9 |

The characterisation and stability results of Formula D are shown in Table 7 below.

TABLE 7
Characterisation and stability results

| | Formula D |
|---|---|
| Macroscopic appearance | Off-white thick milk |
| Microscopic appearance | Fine fractionation, globules <2.5 μm mainly |
| Zeiss Axio Scope A1 | and some rare globules <15 μm |
| Microscope | Isolated adapalene crystals from 2.5 to 15 μm |
| | Slight refraction of beeswax |
| Viscosity Brookfield | RV/S04/5 rpm—at 1 min |
| DVII + Pro | 32,280 cP (80.7%) |
| Physical stability of the | >3 MONTHS |
| formulation (+40° C.) | STABLE at T1M. |
| Chemical stability of the | |
| active ingredients in the | |
| formulation (+40° C.) | |

The prepared formulation D is homogeneous.

### Example 3

### W/O Emulsion in the Presence of a Water-Soluble Pharmaceutically Active Ingredient in Salified Form in Low Concentration

Formula E detailed below is obtained under low shear and in the presence of an emulsifying couple consisting of sorbitan monooleate and macrogol 30 dipolyhydroxystearate (PEG-30 DPHS).

Table 8 below describes a product composition containing a Brimonidine pharmaceutical active ingredient in the form of tartrate, in small amount in the composition (0.5% w/w).

TABLE 8
Formula E which is a W/O composition
adapted for pharmaceutical use according to
the invention comprising a pharmaceutically
active ingredient, in the form of a tartrate salt.

| | % |
|---|---|
| INCI name | w/w* |
| Sorbitan Monooleate | 4.0 |
| Macrogol 30 Dipolyhydroxystearate | 1.0 |
| (PEG-30 DPHS) | |
| Caprylic capric triglycerides | 10.0 |
| Phenoxyethanol | 0.4 |
| Propylene glycol | 5.0 |
| Methyl Parahydroxybenzoate | 0.1 |
| Acrylamide, AMPS dispersion | 4.0 |
| copolymer 40% isohexadecane | |
| Active ingredient Brimonidine as | 0.5 |
| tartrate salt | |
| Water | 75.0 |

The characterisation and stability results of formula E are shown in Table 9 below.

TABLE 9
Characterisation and stability results

| | Formule E |
|---|---|
| Macroscopic appearance | Cream slightly tinted on |
| | the yellow, which flows |
| Microscopic appearance | Fine Fraction globules |
| Zeiss Axio Scope A1 | <10 μm |
| Microscope | |
| Viscosity Brookfield | RV/S05/10 rpm—for 1 |
| DVII + Pro | min. 22,280 cP (55%) |
| Physical stability of the | >1 month |
| formulation (+40° C.) | |

Formulation E is homogeneous and could be prepared.

The Applicant thus proposes a new form database for pharmaceutical application. These W/O emulsions provide a pleasant sensory experience and thus meet the wishes of patients. As a result, such pharmaceutical compositions according to the invention allow good compliance with the treatment and ensure its effectiveness.

This technology is flexible to meet the various requirements of dermatological pathology treatments.

Depending on the compositions and the process applied, formulations are more or less moisturising. The textures are modular and can evolve from a fluid galenic form (milk) to a more compact form (cream). This allows a varied use of these form bases, to treat very diverse pathologies; they are adapted both by the skin condition (ranging from acne, oily skin, to psoriasis, dry and damaged skin, for example) and to both the area and surface of application such as the face, body, hands or feet.

These W/O emulsions allow the integration of pharmaceutically active ingredients, in solubilised or dispersed form, either in aqueous internal phase, in oily continuous phase or simultaneously in both phases. The integration of various active ingredients allows the use of this new base in many pharmaceutical product developments.

### Example 4

### Method for the Preparation of a W/O Emulsion (Placebo) According to the Invention

TABLE 10
Different phases (A and B) of a composition
according to the invention

| | | |
|---|---|---|
| Ingredients | | % |
| [Brand name] | INCI | w/w* |

Phase A

| | | |
|---|---|---|
| SPAN 80 LQ | Sorbitan Oleate | 1.2 |
| CITHROL | Macrogol | 0.3 |
| DPHS SO | Dipolyhydroxystearate 30 | |
| Marcol 82 | Mineral oil | 10.0 |

Phase B

| | | |
|---|---|---|
| SEPINEO ™ | Acrylamide/Sodium | 4.0 |
| P 600 | Acryloydimethyl Taurate | |
| | Copolymer—Isohexadecane | |
| | Polysorbate 80 | |
| Glycerine | Glycerol | 6.0 |
| Methyl paraben | Methyl parahydroxybenzoate | 0.1 |
| Water | Aqua | Qsp 100 |

The method of preparation comprises the following steps:

Heat phase A to about 60° C. until a homogeneous mixture is obtained under magnetic agitation, then allow to cool to room temperature.

Prepare phase B by solubilising methyl paraben in water at 60° C. under magnetic agitation.

After cooling to room temperature, finalise phase B by adding SEPINEO™ P600 in the aqueous phase containing solubilised methyl paraben and glycerol.

Addition under shear with a blade or stator rotor from 700 rpm to 1,000 rpm until homogenisation.

Introduce phase A into phase B at room temperature, or vice versa.

Mix under moderate agitation (50-150 rpm) with a scraper blade until a homogeneous W/O emulsion is obtained, in less than 30 min.

The composition according to the invention thus obtained is thus in the form of a white opaque fluid cream with a viscosity of 60,560 mPa·s (Brookfield RV/S05/5 rpm).

### Example 5

### Method for the Preparation of a W/O Emulsion in the Presence of an Active Ingredient According to the Invention

TABLE 11
Formula E which is a W/O composition adapted for
Pharmaceutical use according to the invention
comprising a pharmaceutically active
ingredient, in the form of a tartrate salt.

| | | |
|---|---|---|
| Ingredients | | % |
| [Brand name] | INCI | w/w* |

Phase A

| | | |
|---|---|---|
| SPAN 80 LQ | Sorbitan Oleate | 4.0 |
| CITHROL | Macrogol | 1.0 |
| DPHS SO | | |
| Miglyol 812N | Caprylic capric triglycerides | 10.0 |
| Phenoxyethanol | Phenoxetol | 0.4 |

Phase B

| | | |
|---|---|---|
| SEPINEO ™ | Acrylamide/Sodium | 4.0 |
| P 600 | Acryloydimethyl Taurate | |
| | Copolymer—Isohexadecane | |
| | Polysorbate 80 | |
| Propylene glycol | Propylene glycol | 5.0 |
| Active ingredient | — | 0.5 |
| Brimonidine as | | |
| tartrate salt | | |
| Methyl paraben | Methyl | 0.1 |
| | parahydroxybenzoate | |
| Water | Aqua | Qsp 100 |

The method of preparation comprises the following steps:

Heat phase A to about 60° C. until a homogeneous mixture is obtained under magnetic agitation, then allow to cool to room temperature.

Prepare phase B by solubilising methyl paraben in water at 60° C. under magnetic agitation.

After cooling, from 45° C., finalise phase B by adding the active ingredient Brimonidine in the form of tartrate salt previously solubilised in propylene glycol, in the aqueous phase containing the solubilised methyl paraben, then add SEPINEO™ P600.

Addition of SEPINEO™ P600 under shear with a blade or stator rotor from 700 rpm to 1,000 rpm until homogenisation.

Introduce phase A into phase B at room temperature, or vice versa.

Mix under moderate agitation (50-150 rpm) with a scraper blade until a homogeneous W/O emulsion is obtained, in less than 30 min.

The composition according to the invention thus obtained is thus in the form of a fluid opaque cream with a viscosity of 22,280 mPa˜s (Brookfield RV/S05/10 rpm).

### Example 6

### Water/Oil (W/O) Emulsions According to the Invention Obtained Using Different Thickeners

TABLE 12
Formula AA which is a cosmetic
W/O composition according to
the invention comprising Xanthan Gum

| INCI name | % w/w* |
|---|---|
| Sorbitan Monooleate | 1.2 |
| Macrogol 30 | 0.3 |
| Dipolyhydroxystearate | |
| (PEG-30 DPHS) | |
| Caprylic capric triglycerides | 10.0 |
| Glycerol | 6.0 |
| Methyl Parahydroxybenzoate | 0.1 |
| Xanthan Gum | 4.0 |
| Water | 78.4 |

TABLE 13
Formula BB which is a cosmetic W/O
composition according to the invention
comprising hydroxypropyl cellulose

| INCI name | % w/w* |
|---|---|
| Sorbitan Monooleate | 1.2 |
| Macrogol 30 | 0.3 |
| Dipolyhydroxystearate | |
| (PEG-30 DPHS) | |
| Caprylic capric triglycerides | 10.0 |
| Glycerol | 6.0 |
| Methyl Parahydroxybenzoate | 0.1 |
| Hydroxypropyl cellulose | 2.0 |
| Water | 80.4 |

TABLE 14
Formula CC which is a cosmetic W/O
composition according to the invention
comprising hydroxyethylcellulose

| INCI name | % w/w* |
|---|---|
| Sorbitan Monooleate | 1.20 |
| Macrogol 30 | 0.30 |
| Dipolyhydroxystearate | |
| (PEG-30 DPHS) | |
| Caprylic capric triglycerides | 10.00 |
| Glycerol | 6.00 |
| Methyl Parahydroxybenzoate | 0.10 |
| hydroxyethylcellulose | 1.25 |
| Water | 81.15 |

TABLE 15
Formula DD which is a cosmetic
W/O composition according to the
invention comprising starch acetate

| INCI name | % w/w* |
|---|---|
| Sorbitan Monooleate | 1.2 |
| Macrogol 30 | 0.3 |
| Dipolyhydroxystearate | |
| (PEG-30 DPHS) | |
| Caprylic capric triglycerides | 10.0 |
| Glycerol | 6.0 |
| Methyl Parahydroxybenzoate | 0.1 |
| Starch acetate (Starch acetate) | 7.0 |
| Water | 75.4 |

TABLE 16
Characterisation and stability results
of formulae AA, BB, CC and DD

| | Formula | Formula | Formula | Formula |
|---|---|---|---|---|
| | AA | BB | CC | DD |
| Macroscopic | Fluid | Fluid | Fluid | Fluid |
| appearance | cream | cream | cream | cream |
| Microscopic | Hetero- | Hetero- | Hetero- | Hetero- |
| appearance | geneous | geneous | geneous | geneous |
| (Zeiss Axio | emulsion | emulsion | emulsion | emulsion |
| Scope A1 | globules | globules | globules | globules |
| Microscope) | <15 μm | <30 μm | <15 μm | <10 μm |
| Viscosity | RV/S04/5 | RV/S04/5 | RV/S04/5 | RV/S04/5 |
| Brookfield | rpm—for | rpm—for | rpm—for | rpm—for |
| DVII + Pro | 1 min. | 1 min. | 1 min. | 1 min. |
| | 13,000 cP | 11,400 cP | 12,120 cP | 14,800 cP |
| | (33%) | (28%) | (30%) | (37%) |
| Stability | >3 month | >3 months | >3 months | >3 months |
| (+40° C.) | | | | |

The above results show that the physical characteristics of the AA, BB, CC and DD formulas are similar. The addition of polyelectrolyte thickeners on a natural basis (Formula AA) or non-polyelectrolyte polymers (Formula BB, CC and DD) provides stable emulsions.

The formulas are stable for at least three months at +40° C., without modification of the physical components, i.e. macroscopic observation, microscopic observation and viscosity.

### Example 7

### Method for the Preparation of a W/O Emulsion According to the Invention

TABLE 17
Different phases (AA and BB) of a composition
according to the invention

| | | |
|---|---|---|
| Ingredients | | % |
| [Brand name] | INCI | w/w* |

Phase AA

| | | |
|---|---|---|
| SPAN 80 LQ | Sorbitan Oleate | 1.2 |
| CITHROL | Macrogol | 0.3 |
| | Dipolyhydroxystearate 30 | |
| Miglyol 812N | Caprylic capric triglycerides | 10.0 |

Phase BB

| | | |
|---|---|---|
| Beauty ST30 | Starch acetate | 7.0 |
| Glycerine | Glycerol | 6.0 |
| Methyl | Methyl parahydroxybenzoate | 0.1 |
| Water | Aqua | Qsp |

The method of preparation comprises the following steps:

Heat the AA phase to about 60° C. until a homogeneous mixture is obtained under magnetic agitation, then allow to cool to room temperature.

Prepare the BB phase by dispersing the starch acetate under shear with a blade or stator rotor from 700 rpm to 1,000 rpm until homogenisation.

Heat to 55° C. to solubilise the methyl paraben, with moderate agitation, then add glycerin.

After cooling the BB phase to room temperature, introduce phase A into phase BB, or vice versa.

Mix under moderate agitation (50-150 rpm) with a scraper blade until a homogeneous W/O emulsion is obtained, in less than 30 min.

The composition according to the invention thus obtained is in the form of a fluid cream with a viscosity of 14,800 mPa·s (Brookfield RV/SO4/5 rpm).

### Example 8

### W/O Emulsion in the Presence of a Water-Soluble Pharmaceutically Active Ingredient in Salified Form in Low Concentration

The Formula AAA detailed below is obtained under low shear and in the presence of an emulsifying couple consisting of sorbitan monooleate and macrogol 30 dipolyhydroxystearate (PEG-30 DPHS).

Table 18 below describes a product composition containing a Brimonidine pharmaceutical active ingredient in the form of tartrate, in small amount in the composition (0.5% w/w).

TABLE 18
Formula AAA which is an W/O composition
adapted for pharmaceutical use according
to the invention comprising a pharmaceutically
active ingredient, in the form of a tartrate salt.

| INCI name | % w/w* |
|---|---|
| Sorbitan Monooleate | 4.0 |
| Macrogol 30 Dipolyhydroxystearate | 1.0 |
| (PEG-30 DPHS) | |
| Caprylic capric triglycerides | 10.0 |
| Phenoxyethanol | 0.4 |
| Propylene glycol | 5.0 |
| Methyl Parahydroxybenzoate | 0.1 |
| Acrylamide, AMPS dispersion | 4.0 |
| copolymer 40% isohexadecane | |
| Active ingredient Brimonidine as | 0.5 |
| tartrate salt | |
| Water | 75.0 |

The characterisation and stability results of the AAA formula are shown in Table 19 below.

TABLE 19

| | Formula AAA |
|---|---|
| Macroscopic appearance | Cream slightly tinted on the |
| | yellow, which flows |
| Microscopic appearance | Fine Fraction |
| Zeiss Axio Scope A1 | globules <10 μm |
| Microscope | |
| Viscosity Brookfield | RV/S05/10 rpm—for 1 min. |
| DVII + Pro | 22,280 cP (55%) |
| Physical stability of the | >1 month |
| formulation (+40° C.) | |

The AAA formulation is homogeneous and could be prepared.

The Applicant thus proposes a new form database for pharmaceutical application. These W/O emulsions provide a pleasant sensory experience and thus meet the wishes of patients. As a result, such pharmaceutical compositions according to the invention allow good compliance with the treatment and ensure its effectiveness.

This technology is flexible to meet the various requirements of dermatological pathology treatments.

Depending on the compositions and the process applied, formulations are more or less moisturising. The textures are modular and can evolve from a fluid galenic form (milk) to a more compact form (cream). This allows a varied use of these form bases, to treat very diverse pathologies; they are adapted both by the skin condition (ranging from acne, oily skin, to psoriasis, dry and damaged skin, for example) and to both the area and surface of application such as the face, body, hands or feet.

These W/O emulsions allow the integration of pharmaceutically active ingredients, in solubilised or dispersed form, either in aqueous internal phase, in oily continuous phase or simultaneously in both phases. The integration of various active ingredients allows the use of this new base in many pharmaceutical product developments.

### Example 9

### Method for the Preparation of a W/O Emulsion (Placebo) According to the Invention

TABLE 20
Different phases (A and B) of a composition
according to the invention

| | | |
|---|---|---|
| Ingredients | | % |
| [Brand name] | INCI | w/w* |

Phase A

| | | |
|---|---|---|
| SPAN 80 LQ | Sorbitan Oleate | 1.2 |
| CITHROL | Macrogol | 0.3 |
| DPHS SO | Dipolyhydroxystearate 30 | |
| Marcol 82 | Mineral oil | 10.0 |

Phase B

| | | |
|---|---|---|
| SEPINEO ™ | Acrylamide/Sodium | 4.0 |
| P 600 | Acryloydimethyl Taurate | |
| | Copolymer—Isohexadecane | |
| | Polysorbate 80 | |
| Glycerine | Glycerol | 6.0 |
| Methyl paraben | Methyl parahydroxybenzoate | 0.1 |
| Water | Aqua | Qsp 100 |

The method of preparation comprises the following steps:

Heat phase A to about 60° C. until a homogeneous mixture is obtained under magnetic agitation, then allow to cool to room temperature.

Prepare phase B by solubilising methyl paraben in water at 60° C. under magnetic agitation.

After cooling to room temperature, finalise phase B by adding SEPINEO™ P600 in the aqueous phase containing solubilised methyl paraben and glycerol.

Addition under shear with a blade or stator rotor from 700 rpm to 1,000 rpm until homogenisation.

Introduce phase A into phase B at room temperature, or vice versa.

Mix under moderate agitation (50-150 rpm) with a scraper blade until a homogeneous W/O emulsion is obtained, in less than 30 min.

The composition according to the invention thus obtained is thus in the form of a white opaque fluid cream with a viscosity of 60,560 mPa·s (Brookfield RV/S05/5 rpm).

### Example 10

### Method for the Preparation of a W/O Emulsion in the Presence of an Active Ingredient According to the Invention

TABLE 21
Formula A which is an W/O composition
adapted for pharmaceutical use according
to the invention comprising a pharmaceutically
active principle, in the form of a tartrate salt.

| | | |
|---|---|---|
| Ingredients | | % |
| [Brand name] | INCI | w/w* |

Phase A

| | | |
|---|---|---|
| SPAN 80 LQ | Sorbitan Oleate | 4.0 |
| CITHROL | Macrogol | 1.0 |
| DPHS SO | Dipolyhydroxystearate 30 | |
| Miglyol 812N | Caprylic capric triglycerides | 10.0 |
| Phenoxyethanol | Phenoxetol | 0.4 |

Phase B

| | | |
|---|---|---|
| SEPINEO ™ | Acrylamide/Sodium | 4.0 |
| P 600 | Acryloydimethyl Taurate | |
| | Copolymer—Isohexadecane | |
| | Polysorbate 80 | |
| Propylene glycol | Propylene glycol | 5.0 |
| Active ingredient | — | 0.5 |
| Brimonidine as | | |
| Methyl paraben | Methyl parahydroxybenzoate | 0.1 |
| Water | Aqua | Qsp |

The method of preparation comprises the following steps:

Heat phase A to about 60° C. until a homogeneous mixture is obtained under magnetic agitation, then allow to cool to room temperature.

Prepare phase B by solubilising methyl paraben in water at 60° C. under magnetic agitation.

After cooling, from 45° C., finalise phase B by adding the active ingredient Brimonidine in the form of tartrate salt previously solubilised in propylene glycol, in the aqueous phase containing the solubilised methyl paraben, then add SEPINEO™ P600.

Addition of SEPINEO™ P600 under shear with a blade or stator rotor from 700 rpm to 1,000 rpm until homogenisation.

Introduce phase A into phase B at room temperature, or vice versa.

Mix under moderate agitation (50-150 rpm) with a scraper blade until a homogeneous W/O emulsion is obtained, in less than 30 min.

The composition according to the invention thus obtained is thus in the form of a fluid opaque cream with a viscosity of 22,280 mPa·s (Brookfield RV/S05/10 rpm).

1. A water-in-oil (W/O) emulsion composition, comprising:
(a) 60% to 98%, by weight of the composition, an aqueous phase, and
(b) a fat phase comprising one or a plurality of oils and an emulsifying system comprising a sorbitan ester and a polyglycerol ester.

2. The composition according to claim 1, wherein the composition is a pharmaceutical composition suitable for topical administration and further comprises at least one pharmaceutically active ingredient; and wherein the aqueous phase is an aqueous gel phase and further comprises a polyelectrolyte rheology modifier, a non-polyelectolyte rheology modifier, or a combination thereof.

3. The composition according to claim 2, wherein the composition is free of octyldodecanol and/or octyldodecylxyloside.

4. The composition according to claim 2, wherein the pharmaceutically active ingredient is selected from the group consisting of antibiotics, antibacterial agents, antivirals, antiparasitic agents, antifungals, anaesthetics, analgesics, painkillers, antiallergic agents, antiacneics, antimitotics, antipruritic drugs, antihistamines, immunosuppressants, corticosteroids, keratolytics, anti-angiogenics, anti-inflammatory drugs, phosphodiesterase 4 inhibitors, anti-cancer drugs, anti-neoplastic drugs, anthracene derivatives, psoralens, anti-proliferative drugs, vitamin D analogues, anti-alopecics (prostaglandin analogues), anti-herpetics, photosensitisers, depigmentants, hormones, retinoids, vasoconstrictors, and a mixture or two more thereof.

5. The composition according to claim 2, wherein the pharmaceutically active ingredient is selected from the group consisting of acetaminophen, acefylsalicylic acid, acifrefin, azelaic acid, acyclovir, adapalene, alclomefasone, alpha-tocopherol, amcinonide, amorolfine, amphotericin B, tetracycline, benzoyl peroxide, betamethasone, brimonidine, calcipotriol, calcitriol, ciclopirox, clindamycin, crisaborole, clobetasol, crotamiton, cyproheptadine, dapsone, desonide, diclofenac, diflucortolone, difluprednafe, dioxyanthranol, econazole, efinaconazole, erythromycin, estradiol, etretinate, fluocinolone acetonide, fluticasone, fusidic acid, momefasone, glycolic acid, glycyrrhefinic acid, halobefasol, hydrocortisone, hydroquinone, ibuprofen, imiquimod, isotretinoin, ivermectin, kefoconazole, kojic acid, lactic acid, lidocaine, malic acid, mequinol, mefhoxsalene, metronidazole, miconazole, minoxidil, ocfopirox, oxymefazoline, pilocaine, pyridoxine, progesterone, retinol, pimecrolimus, resiquimod, rucinol, tacrolimus, tazarofene, terbinafine, tetracaine, thenaldine, fravopost, tretinoin, trimeprazine, trimeprazine, trifarofene, zinc pyrithione, and salts or derivatives of these active ingredients, and a mixture of two or more thereof.

6. The composition according to claim 2, wherein the polyelectrolyte rheology modifier is selected from synthetic polymers and polymers of natural origin.

7. The composition according to claim 6, wherein the synthetic polymers and the polymers of natural origin are selected from the group consisting of copolymers of acrylic acid and 2-methyl-[(1-oxo-2-propenyl)amino]1-propane sulfonic acid (AMPS), copolymers of acrylamide and 2-methyl-[(1-oxo-2-propenyl)amino]1-propane sulfonic acid, copolymers of 2-methyl-[(1-oxo-2-propenyl)amino]1-propane sulfonic acid and (2-hydroxyethyl) acrylate, a homopolymer of 2-methyl-[(1-oxo-2-propenyl)amino]1-propane sulfonic acid, a homopolymer of acrylic acid, copolymers of acryloyl ethyl trimethyl ammonium chloride and acrylamide, copolymers of AMPS and vinylpyrrolidone, copolymers of acrylic acid and alkyl acrylates containing ten and thirty carbon atoms, copolymers of AMPS and alkylacrylates containing ten and thirty carbon atoms, gelatin, carrageenans, pectins, alginates, agarose, agar-agar, chitosan, and xanthan gum.

8. The composition according to claim 2, wherein the non-polyelectrolyte rheology modifier is selected from a non-polyelectrolyte polymer.

9. The composition according to claim 8, wherein the non-polyelectrolyte rheology modifier is a synthetic non-electrolyte polymer comprising polymerized monomers having a vinyl group.

10. The composition according to claim 1, wherein the sorbitan ester is sorbitan monooleate.

11. The composition according to claim 10, wherein the polyglycerol ester is macrogol 30 dipolyhydroxysfearafe.

12. The composition according to claim 11, wherein the sorbitan ester and the polyglycerol ester are present in a ratio between 2:1 and 10:1.

13. The composition according to claim 12, wherein the sorbitan monooleate is present at no more than 4 wt% of the total composition.

14. The composition according to claim 1, wherein the polyglycerol ester is macrogol 30 dipolyhydroxysfearafe.

15. The composition according to claim 1, wherein the sorbitan ester and the polyglycerol ester are present in a ratio between 2:1 and 10:1.

16. The composition according to claim 15, wherein the sorbitan ester and the polyglycerol ester are present in a ratio between 3:1 and 5:1.

17. The composition according to claim 1, wherein the sorbitan ester is present at no more than 4 wt% of the total composition.

18. The composition according to claim 10, wherein the sorbitan ester is present at no more than 4 wt% of the total composition.

19. A method of preventing and/or treating a dermatological disease comprising topically administering the composition according to claim 2 to a subject's skin in need thereof.

20. A method for preparing the composition according to claim 2 comprising:
preparing the fat phase;
preparing the aqueous phase;
adding the fat phase to the aqueous phase or adding the aqueous phase to the fat phase; and
recovering the composition.
